(19) United States
(12) Patent Application Publication
(10) Pub. No.: US 2026/0248558 A1
(43) Pub. Date: Aug. 27, 2026

(54) AUTOMATED SYSTEMS AND METHODS FOR ASSISTING WITH MEDICAL PROCEDURES

(71) Applicant: BOSTON SCIENTIFIC SCIMED, INC., Maple Grove, MN (US)

(72) Inventor: Michael F. McGovern, San Diego, CA (US)

(73) Assignee: BOSTON SCIENTIFIC SCIMED, INC., Maple Grove, MN (US)

(21) Appl. No.: 19/449,989

(22) Filed: Jan. 15, 2026

Related U.S. Application Data
(60) Provisional application No. 63/746,162, filed on Jan. 16, 2025.

(51) Int. Cl. A61B 34/10; A61B 1/00; A61B 1/273; A61F 5/00; A61B 17/04; A61B 34/00; A61B 34/20

(52) U.S. Cl. CPC A61B 34/10; A61B 1/00009; A61B 1/00045; A61B 1/2736; A61F 5/0083; A61F 5/0089; A61B 17/0469; A61B 2034/107; A61B 2034/2065; A61B 2034/252

(57) ABSTRACT

An automated system for assisting a medical professional with performing a medical procedure within a patient’s body, such as an endoscopic procedure which does not provide direct visualization of the patient’s anatomy. The automated system is trained to track and/or predict points, lines, regions, etc., along the patient’s anatomy, even as the contour of the anatomy changes as a result of the medical procedure. The automated system may be advantageously used to perform procedures, such as Endoscopic Sleeve Gastroplasty procedures, to address challenges presented by the anatomy. The automated system may assist in suture placement, and/or maintaining orientation with respect to the anatomy, and/or providing guidance in performing the procedure by predicting actions to be taken during the procedure (e.g., recommending suture placement), and/or providing notifications of unintended and/or undesired configurations of the anatomy or structures positioned with respect to the anatomy.

### CROSS-REFERENCE TO RELATED APPLICATIONS

This application claims the benefit of priority under 35 U.S.C. § 119 to U.S. Provisional Application No. 63/746,162, filed on January 16, 2025, the entire disclosure of which is hereby incorporated by reference herein for all purposes.

### FIELD

The present disclosure relates generally to the field of medical devices, systems, and methods for performing a procedure within a patient’s body. More particularly, the present disclosure relates to systems and methods for performing procedure in which medical professionals have limited direct visibility, such as endoscopic procedures, and which utilize electronic imaging systems to assist the medical professional in viewing the treatment site.

### BACKGROUND

Minimally invasive procedures, in contrast with open surgical procedures, do not require open surgery, and include procedures such as percutaneous and/or transluminal (e.g., endoscopic) procedures. Typically, medical professionals who perform minimally invasive procedures, rely on a visualization system inserted into the patient’s body. Without a direct view of the treatment site within the patient’s body, the medical professional may face various challenges. One example of an endoscopically performed procedure which presents significant challenges is an Endoscopic Sleeve Gastroplasty (ESG) procedure. The complexity of the procedure, particularly the suturing process, demands extensive training (especially at least for the medical professional’s first fifty or so procedures). The medical professionals must develop muscle memory, such as for precise suturing, and must master complex suturing techniques. The mechanical aspects of the procedure, alone, contribute to a significant learning curve. Moreover, approximately 75% of the procedure time is spent determining suture placement and orientation within the stomach, as the medical professional struggles to navigate and interpret the view of the treatment site on the video display (e.g., screen or monitor) which provides an automated image of the treatment site within the patient’s body. Once suturing has begun, scope movability is immediately constrained by the connection of the scope to the treatment site via the suture. Moreover, the patient’s anatomy changes shape as the procedure is being performed, presenting further challenges. For instance, the changing anatomy may make it extremely difficult for the medical professional to orient and determine anatomical context. Medical professionals may thus find it difficult to identify the exact location for sutures due to limited anatomical orientation and tethering effects after the first stitch. Such challenges significantly increase procedure time and complexity. Solutions to these and other challenges in the art would therefore be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an automated system is configured to assist with performance of a medical procedure with respect to a treatment site along the anatomy of a patient. In some aspects, the system includes an image processor operably associated with a medical instrument having an imaging device, the image processor configured to receive and process signals from the imaging device to identify a point, line, region, or object along a patient’s anatomy, and to predict movement of the point, line, region, or object along the patient’s anatomy as the patient’s anatomy is modified during the course of a medical procedure; and a video display unit operably associated with the image processor to display the point, line, region, or object to the medical professional in real-time while the medical professional performs the medical procedure.

In some aspects, the image processor is configured to output a representation of markings input by the medical professional.

In some aspects, the image processor is configured to output a representation of markings of one or more lines of sutures the medical professional intends to make along the anatomy of the patient, and tracks the representation of the markings as the medical professional performs the medical procedure.

In some aspects, the image processor is configured to output a representation of points at which the medical professional intends to suture along the treatment site.

In some aspects, the image processor is configured to output a representation of directions and/or landmarks to orient the medical professional as the patient’s anatomy is modified during the course of the medical procedure. In some aspects, the output of the image processor is based on markings input by the medical professional indicating anatomical directions at the treatment site and/or anatomical landmarks at or near the treatment site. In some aspects, the output of the image processor is an indication of a direction in which a point, line, or region of the anatomy is located, the point, line, or region not being visually displayed by the video display unit. In some aspects, the point, line, or region is input by the medical professional prior to commencing the procedure and/or during the medical procedure.

In some aspects, the image processor is configured to output an indication of an undesirable and/or unintended configuration occurring during the medical procedure. In some aspects, the output of the image processor is an indication of a kink in a suture used during the medical procedure.

In accordance with various principles of the present disclosure, an automated system is configured to assist with surgical procedures within a patient’s stomach. In some aspects, the system includes an image processor operably associable with an imaging device of an endoscope and configured to receive and process signals from the imaging device to identify a point, line, region of the patient’s stomach and to predict movement of the point, line, or region, or an object along the patient’s stomach as the configuration of the patient’s stomach is modified during the course of a medical procedure; and a video display unit operably associated with the image processor to display the point, line, region, or object to the medical professional in real-time while the medical professional performs the medical procedure.

In some aspects, the image processor is configured to output a representation of markings of one or more lines of sutures the medical professional intends to make along the patient’s stomach, and to track the representation of the markings as the medical professional performs the medical procedure and the contours of the stomach are modified by the medical procedure.

In some aspects, the image processor is configured to output a representation of points at which the medical professional intends to suture along the stomach to modify the shape or volume of the stomach, and to track the representation of the points as the medical professional performs the medical procedure and the contours of the stomach are modified by the medical procedure.

In some aspects, the image processor is configured to output a representation of directions and/or landmarks within the patient’s stomach to orient the medical professional as the patient’s stomach is modified during the course of the medical procedure, and/or to direct the medical professional in the direction and/or landmark if not represented on the video display unit.

In some aspects, the image processor is configured to output an indication of a kink in a suture and/or an undesirable and/or unintended configuration of the stomach occurring during the medical procedure.

In accordance with various principles of the present disclosure, a method of providing automated assistance during performance of an endoscopic sleeve gastroplasty procedure includes providing an image processor trained to track points, lines, regions, or objects, along a stomach lining of a patient and operably associable with an imaging device of an endoscope; and providing a video display unit for displaying images from the imaging device of the endoscope processed by the image processor, including virtual representations of points, lines, regions, or objects of interest to the medical professional for performing the endoscopic sleeve gastroplasty procedure.

In some aspects, the method further includes providing a user interface for the medical professional to input points, lines, or regions of interest to track and to display on the video display unit during the procedure.

In some aspects, providing an image processor further includes providing an image processor trained to predict and recommend guidelines for suturing the patient’s stomach to reduce the volume of the patient’s stomach.

In some aspects, providing an image processor further includes providing an image processor trained to notify the medical professional of a kink in a suture and/or an undesirable and/or unintended configuration of the stomach occurring during the endoscopic sleeve gastroplasty procedure.

In some aspects, providing an image processor further includes providing an image processor trained to indicate anatomical directions and/or landmarks within the stomach as the configuration of the stomach changes during the endoscopic sleeve gastroplasty procedure.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an example of an embodiment of an automated system formed in accordance with aspects of the present disclosure.

FIG. 2 illustrates a perspective view of a medical device which may be used in performing a procedure to be assisted by an automated system such as illustrated in FIG. 1.

FIG. 3 schematically illustrates a training process in accordance with various principles of the present disclosure which may be used to track points, lines, regions, etc., within a patient.

FIG. 4 schematically illustrates tracking capabilities of an automated system formed in accordance with various principles of the present disclosure,

FIG. 5 schematically illustrates guidelines or prediction capabilities of an automated system formed in accordance with various principles of the present disclosure.

FIG. 6 schematically illustrates an anatomical compass feature of an automated system formed in accordance with various principles of the present disclosure.

FIG. 7 schematically illustrates an anatomical compass feature with directional indicators of an automated system formed in accordance with various principles of the present disclosure.

FIG. 8 schematically illustrates a feature detection system of an automated system formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All systems and methods discussed herein are examples of systems and/or methods implemented in accordance with one or more principles of this disclosure. Each example of an embodiment is provided by way of explanation and is not the only way to implement these principles but are merely examples. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure provides systems and methods for facilitating performance of procedures within a patient’s body with the use of a visualization system (typically an automated visualization system, which may have a narrow or limited field of view) rather than direct visualization by the medical professional. The systems and methods described herein provide real-time automated guidance to improve the ability of the medical professional to navigate within the patient and/or to perform the procedure. The systems and methods may be used with procedures performed with a medical scope with a built-in or accompanying or otherwise integrated automated visualization system.

One particular example of a procedure which may benefit from various principles of the present disclosure is an endoscopic sleeve gastroplasty (ESG) procedure. During an ESG procedure, a medical professional navigates a medical scope (e.g., endoscope) with a suturing device into the patient’s stomach. The medical scope typically has a patient-contacting flexible elongate member which is inserted into the patient. Such flexible elongate member is typically in the form of an elongated tube with a working channel / hollow section defined therethrough (and which may be known as an insertion tube). The working channel is typically sized for various medical devices to be advanced therethrough and into the patient, such as to perform the procedure and/or to take samples. It will be appreciated that terms such as medical devices, instruments, tools, accessories, materials, etc., may be usable interchangeably herein without intent to limit unless otherwise specified. The medical scope typically has, at its proximal end, a control handle which may be used to advance the insertion tube within the patient’s body or to retract the insertion tube back out of the patient’s body. The handle typically includes control knobs which may be used to navigate the medical scope within the patient’s body, and/or to control other accessories of the medical scope, such as irrigation, suction, light, and imaging devices / a visualization system. The control handle includes one or more connectors, such as to supply irrigation fluid, suction pressure, energy, etc., to the medical scope, and/or to connect the visualization system with external accessories, such as a video processor unit and/or a screen / video monitor.

Typically, the medical professional fills the stomach (typically with a gas, such as air) to expand the internal cavity of the stomach, and to reduce the internal folds along the interior of the stomach or otherwise to unfold the stomach wall (which is typically folded around itself). Insufflation of the stomach allows the medical professional to view the total volume and surface area of the stomach. The medical professional then marks two guidelines along the interior lining (mucosa) of the stomach as guidelines along which the medical professional will suture to decrease the volume of the stomach. Marking may be performed by a mechanical means (e.g., a small marking device or marking material such as biocompatible ink) or by otherwise modifying the tissue (e.g., such as burning dots on the stomach lining, such as with argon gas, or another cauterization device). The medical professional determines intended suture points along the guidelines, and then proceeds with suturing along the guidelines. To insufflate, mark, and suture the stomach, the medical scope must be repeatedly advanced closer to the treatment site (with a relatively narrow field of view) and retracted away from the treatment site (for a wider view of the treatment site and its surrounding anatomy). Such repeated change of the field of view provided by the visualization system of the medical scope may disorient the medical professional. Additionally, the nature of the gastric environment also presents various challenges in identifying points, lines, regions, etc., along the stomach lining. For instance, illumination, light reflectivity and/or glare effects, the limited color differentiation of the tissue, possible occlusions (e.g., from fluids, tools, or anatomy that blocks the view), and/or other subtle differences, distinctions, gradations, etc., in the surface of the stomach line provide initial challenges to the medical professional.

As soon as the first suture has been placed, the medical scope becomes limited in its possible movement by the end of the suture secured to the tissue. The usual manipulations of the control knobs may not result in the expected or desired articulation and/or directional movement because the medical scope is now tethered to the treatment site by the suture. As the medical professional navigates the suturing device within the patient’s body to remaining points at which tissue is to be sutured, the portion of the suture which has been extended into tissue continues to affect movement and navigation of the medical scope, limiting the degree of freedom of movement of the medical scope. Moreover, the anatomy of the stomach shifts and is reconfigured as the medical professional continues to suture the stomach to reconfigure the stomach. The continuous motion and changing of the shape of the stomach (including deformation of the stomach) throughout the procedure may cause the medical professional to lose his or her orientation within the patient and relative to a frame of reference at the treatment site. As the medical professional tightens the suture, the medical professional must constantly examine the live images provided by the visualization system for potential suture kinks. Once the suturing has been completed, the medical professional tightens the suture to draw together the tissue and reshape the stomach into the final smaller configuration intended by the ESG procedure. A suture cinch or anchor is placed to secure the suture in place with the stomach reconfigured as desired / medically indicated.

In accordance with various principles of the present disclosure, the various challenges presented by the limited degrees of freedom of movement of the medical scope, and the shifting shape and contours of the anatomy are alleviated by the use of an automated, digitally-assisted / artificial intelligence system designed to track a point or object within a patient’s body, such as displayed on a video display unit operably associated with the medical scope’s visualization system. The particular tracking system is not critical, and any off-the-shelf system may be used, and trained in accordance with various principles of the present disclosure to be used in accordance with various principles of the present disclosure. The systems and methods of the present disclosure provide automated, real-time assistance in a variety of aspects of a minimally-invasive procedure performed within a body without direct visibility to the medical professional, and/or as the medical professional struggles with limited device mobility and changing anatomy after the procedure has begun (e.g., limited medical scope mobility during suturing). For instance, systems and methods of the present disclosure provide real-time computer-assisted navigation and tracking during a medical procedure (generally, a procedure performed without direct visibility to the medical professional performing the procedure) such as by digitally tracking points, lines, regions, etc., of the anatomy marked or otherwise identified or selected by the medical professional and with respect to which the procedure is to be performed. For instance, points such as suture points; lines such as suture lines or guidelines; regions such as anatomical features or landmarks, may be marked, tracked, manipulated, or otherwise input and processed by a system of the present disclosure. Additionally or alternatively, the systems and methods of the present disclosure may also assist with predicting optimal steps and/or next steps in a medical procedure (e.g., suture placement) and/or detecting potential issues arising during the medical procedure (e.g., suture kinks). Additionally or alternatively, systems and methods of the present disclosure may provide guidance during a medical procedure in which the landscape is continuously modified by the procedure. For instance, the systems and methods of the present disclosure may monitor, track, and/or provide a digital representation of the landscape of the treatment site (points and/or surface features, contours, characteristics, etc.) as the treatment site is modified by the procedure.

The assistance provided by systems and methods of the present disclosure address the primary challenges of various minimally-invasive procedures (such as ESG), such as determining the steps to be taken (upon viewing the treatment site prior to or while performing the procedure, such as determining suture placement) and/or determining orientation of the instruments with respect to the anatomy, such as for navigation purposes. Systems and methods of the present disclosure may be used to determine placement of the instruments (e.g., to perform a procedure), deployment of materials or devices (e.g., sutures, anchors, staples, etc.), and general orientation within the patient’s body (such as to assist with navigation within the patient’s body).

Various systems and methods of the present disclosure will now be described with reference to examples illustrated in the accompanying drawings. It should be understood that one or more of the features, structures, concepts, and/or characteristics described herein with reference to one aspect of the disclosure can be combined with one or more of the features, structures, concepts, and/or characteristics of any of the aspects described herein. That is, any of the features, structures, concepts, and/or characteristics described herein can be mixed and matched to create hybrid solutions, and such hybrid solutions are within the scope of the present disclosure. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

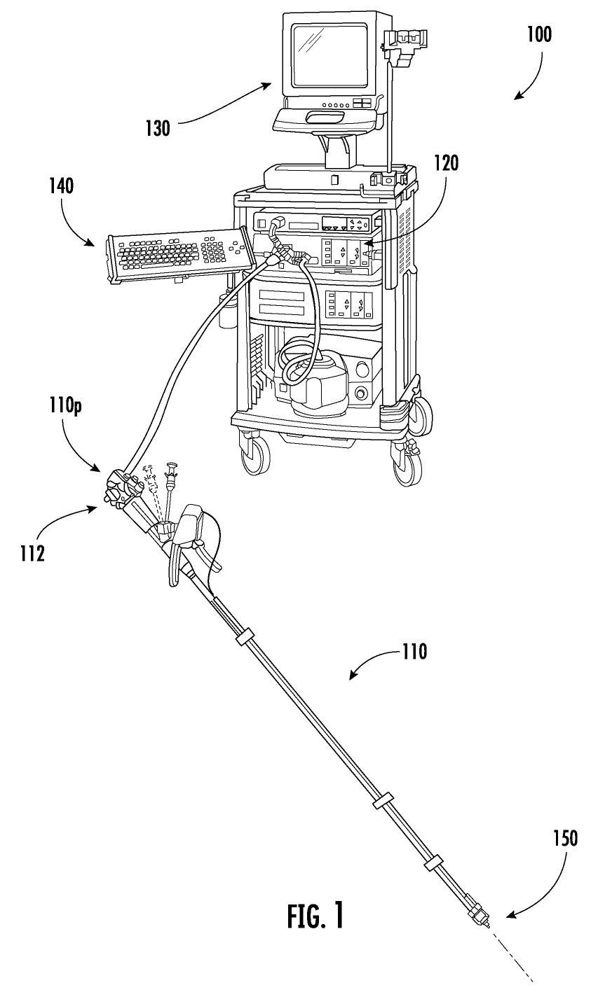

Turning now to the drawings, an example of an embodiment of a system 100 formed in accordance with various principles of the present disclosure for performing a medical procedure is illustrated in FIG. 1. The system 100 includes a medical instrument 110 including members, features, components, etc., configured to access a treatment site within the patient, and/or to engage a treatment site, and/or to provide access to or for another instrument which engages the treatment site. In the illustrated example of an embodiment, the medical instrument 110 is an endoscope, although other medical instruments are usable with the system 100. A control handle 112 typically is provided at the proximal end 110p of the medical instrument 110 with one or more user interface components (e.g., knobs, levers, etc.) configured for engagement (e.g., manual, such as grasping) and movement by the medical professional to operate, control, manipulate, etc., one or more components of or operably associated with the medical instrument 110, such as in a manner known to those of ordinary skill in the art.

In accordance with various principles of the present disclosure, the system 100 includes a processor 120, such as a computer or any other processor, configured and trained to process data input into the system 100, and to convert the data into information usable by the medical professional, such as before, during, and/or after the procedure. More particularly, the processor 120 may execute a trained model, such as a neural network, which performs computations and analyses to provide information usable by a medical professional in performing a procedure. Even more particularly, the information may be visual information of anatomical features not readily directly viewable by the medical professional, such as internal anatomical features viewed by a medical scope inserted into the patient in a minimally invasive manner (i.e., not through open surgery, but through a natural orifice or small incision). In some aspects, the processor 120 is an image processor 120 capable of being operably associated with an imaging device operably associated with the system 100. In some aspects, the medical instrument 110 includes an imaging device / system (e.g., camera, fiber optic, etc., such as known or heretofore known to those of ordinary skill in the art configured for visualizing the patient’s anatomy) operably associated with (e.g., providing data to) the image processor 120. In some aspects, the image processor 120 includes, without limitation, memory, input/output peripherals or interfaces, storage (e.g., internal, long-term storage for machine learning capabilities), and one or more computing / processing units. The computing / processing units may include one or more of the following, without limitation: a central processing unit (CPU) capable of handling general-purpose automated processing of data; a graphics processing unit (GPU) capable of processing and optionally optimizing graphical data; a neural processing unit (NPU) capable of performing and optionally accelerating machine learning tasks; a tensor processing unit (TPU) capable of performing tensor computations, such as for deep learning with respect to multi-dimensional structures; and/or other hardware units configured to provide additional computational output which may be used to accelerate machine learning tasks. In some aspects, output from the image processor 120 is visually displayed for the medical professional on, for example, a video display unit 130. The video display unit 130 may be in any of a variety of configurations known or heretofore known by one of ordinary skill in the art configured to display visual images or video from within the patient for viewing by the medical professional performing the procedure and/or to display other data to assist the medical professional in performing the procedure. It will be appreciated that the term video display unit is used broadly herein, and intended to encompass any form of monitor, screen, display, etc., unit and/or system by which the medical professional may obtain a preferably real-time image of the treatment site. The illustrated system 100 also includes a user interface 140 by which the medical professional may input data, commands, etc., into the system 100. The user interface 140 may include, among other devices, a keyboard, buttons, joysticks, microphones, etc., configured for engagement and interaction by a user for intake of data, commands, etc., by the system 100, such as by the image processor 120, and/or a user interface (e.g., a button, joystick, trackball, etc.) which may be used to digitally mark a spot or area on tissue (such as relative to the distal end of a medical scope), such as known to those of ordinary skill in the art. It will be appreciated that the video display unit 130 may incorporate a touch screen as another form of a user interface for the system 100.

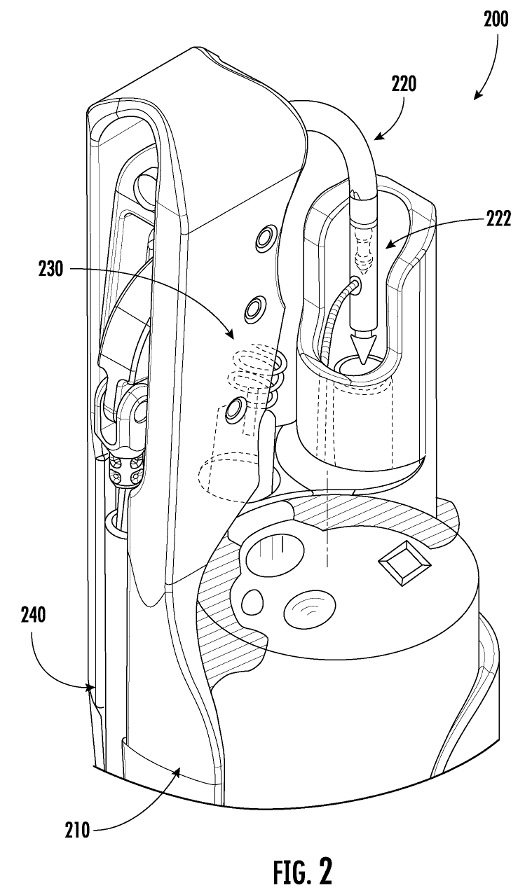

Although the systems and methods described herein may be used in a variety of medical procedures, the present disclosure addresses and provides solutions for, as a non-limiting example, the numerous challenges presented by ESG procedures. An example of an embodiment of a suturing device 200 usable to perform an ESG procedure, and incorporable into the example of an embodiment of a system 100 illustrated in FIG. 1, is illustrated in FIG. 2. The illustrated suturing device 200 includes a mount 210 configured to be operably coupled with the medical instrument 110 (e.g., the insertion tube of an endoscope, such as the distal end thereof). Extending distally from the mount 210 are the operable components of the suturing device 200, which include a needle assembly 220 operably coupled to the mount 210 to be movable to pass a needle 222 through tissue. Optionally, the suturing device 200 also includes a tissue grasper 230 configured to grasp and pull tissue into the pathway of the needle 222. A transmission assembly 240 may operably associate a control handle (e.g., the control handle 112 of the medical instrument 110 or another control handle, preferably in proximity thereto) with one or more components of the suturing device 200 so that the medical professional may operate, control, manipulate, etc., one or more components of the suturing device 200. Various further details, features, components, etc., of a suturing device which may be used in a system such as described herein are described in one or more of the following patent publications which are incorporated herein by reference in their entireties and for all purposes: U.S. Patent 8,287,556, issued October 16, 2012; U.S. Patent 9,867,610, issued January 16, 2018; U.S. Patent 9,198,562, issued December 1, 2015; U.S. Patent 11,141,147, issued October 12, 2021; and Patent Application Publication 2024/0366213, published November 7, 2024.

In some aspects, a system 100 formed in accordance with various principles of the present disclosure allows a medical professional to virtually or digitally mark (in contrast with physically mark, such as with a mechanical device and/or with a material which physically engages tissue) a point, line, or region, etc., within a patient’s body. Additionally or alternatively, in some aspects, a system 100 formed in accordance with various principles of the present disclosure tracks a point, line, or region, etc., marked or otherwise indicated by the medical professional and/or a point, line or region, etc., suggested by the system 100. It will be appreciated, in view of the above, that reference herein to a “region” is to be interpreted broadly, such as encompassing an area, landmark, or other anatomical feature. In order for the system 100 to be able to track a point, line, or region, etc., the image processor 120 may utilize or execute any of a variety of open source or off-the-shelf programs or models, including, without limitation, neural networks, which may use point and/or pattern tracking algorithms, such as algorithms using TAPIR (Tracking Any Point with per-frame Initialization and temporal Refinement), and which have been trained in accordance with various principles of the present disclosure. Algorithms such as those using TAPIR allow the program or model of the image processor 120 to provide advanced, machine-learning-based tracking which efficiently processes and tracks the patient’s anatomy in real-time, even as the anatomy morphs during a procedure. The image processor 120 is capable of managing and processing large amounts and streams of data in parallel, ensuring reliable and scalable tracking of the anatomy comprising one or more (e.g., many) digitally marked points during the procedure which is being performed with respect to the anatomy.

As may be appreciated, in view of the above-described procedural and anatomical challenges presented by performing medical procedures within a patient’s body, the model used by a system and method of the present disclosure must be trained to detect, differentiate, follow, etc., a particular point, line, or region, etc., along the patient’s anatomy. For instance, the model of a tracking program used in a system of the present disclosure is trained, in accordance with various principles of the present disclosure, on proprietary medical images to be able to provide the desired output useful to the medical professional in performing the medical procedure. The training process may involve data collection and pre-processing steps; inputting of data and analysis of predictions based on the input; utilization of one or more specialized algorithms; and development of models (e.g., model architectures) to train, optimize, and generalize the performance of a neural network to be used in processing (e.g., marking, tracking, predicting, etc.) medical images.

Initially, to begin training of the program or model to be used by a system formed in accordance with various principles of the present disclosure to virtually track and/or to provide other guidance with respect to an area of an anatomical structure, an anatomical structure is digitally scanned for an initial point of reference, such as to create a training video. The anatomical structure may be continuously scanned thereafter, as a procedure is performed, to collect data for training purposes. A large data set, from as large an available population as possible, is needed for proper training of the tracking program or model given the challenges the anatomy presents, such as, without limitation, in terms of contour (including changes in contour occurring during the procedure), light reflectivity / glare, monochromatic surfaces, the flexibility and/or deformability of the tissue, occlusions (e.g., fluids, tools, anatomy, etc., that blocks the view), continuous motion / movement, etc., and other subtle differences, distinctions, gradations, etc., in the surface of the anatomical structure. For instance, tens of thousands of images may be gathered for input into the model for training purposes. A model with pretrained weights for general purpose point training (e.g., after having been trained on millions of web images) may be fine-tuned using hundreds or thousands of images for specific use in medical data point tracking.

In some aspects, the expertise of a medical professional, such as in recognizing features, landmarks, etc., of the anatomy, is needed to provide and/or validate the ground truths for the training routine. For instance, a medical professional may provide annotations of the initial point and all subsequent points in the training dataset used for training a model to track an anatomical point, feature, structure, etc. In some aspects, the training dataset is batches of frames of a training video. Images of an anatomical structure (e.g., video images, such as frame by frame, of a training video) may be annotated by one or more medical professionals. For instance, a medical professional may annotate a point of interest to track across multiple frames of a training video, such as by drawing ground truth annotations (e.g., boxes, x’s, circles, etc.) on every video frame. The annotations may be used as reference points / ground truth data within the training set used by the model of the system 100 (e.g., of the image processor 120) of the present disclosure during training. The model makes predictions, and compares the predictions with the reference points of the annotated frames (used as ground truths in the training process, such as in a ground truth dataset) to develop and fine-tune the model’s ability to predict a point to be tracked.

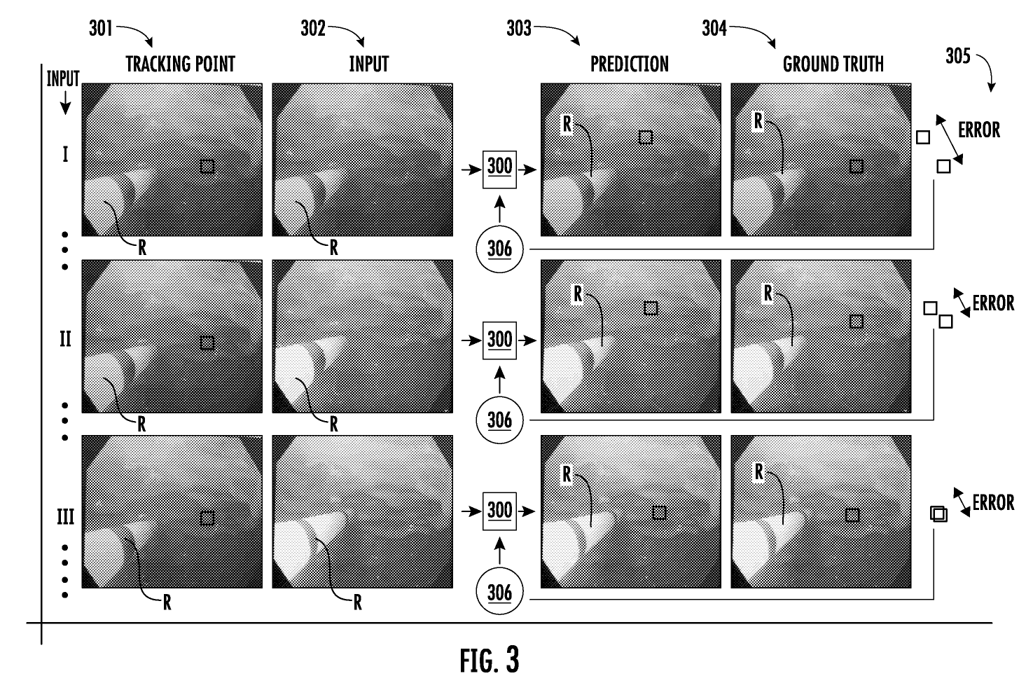

An example of a training routine for a model 300 (e.g., neural network) which may be executed by a processor (e.g., image processor 120 of FIG. 1) of a system of the present disclosure is schematically illustrated in FIG. 3. The training routine teaches the model 300 how to recognize, identify, track, etc., a tracking point 301 identified, for training purposes, in a frame of a batch of frames of a training video of an anatomical structure. The tracking point 301 may be an anatomical feature in the training video frame (e.g., a point, line, region, feature, etc., in the anatomical landscape shown in the frame) represented in FIG. 3 by a box. The training routine is designed to train the model 300 to differentiate variations in microstructures of an anatomical structure by training the model 300 to identify the tracking point 301 in an unannotated input frame 302 (a frame of the training video without the tracking point marked, and generally without any other annotations). For instance, the training routine presents a tracking point 301 along with an unannotated input frame 302 to the model 300 for the forward pass stage of training. It will be appreciated that, in some aspects, the training routine teaches the model 300 to recognize and differentiate the tracking point 301 regardless of the relative position of the tracking point 301 in the frame. As such, the video frame in which the tracking point 301 is presented may be the same in each iteration I, II, III, etc., of the training routine, even as the frame of the input 302, the prediction 303, and the ground truth 304 may change (such as described in further detail below). For instance, in each iteration I, II, III, etc., of the training routine, the tracking point 301 may be presented in the first frame of the training routine, whereas a different frame of the training video may be used in the input 302, the prediction 303, and the ground truth 304. A reference R (e.g., a tool, instrument, or virtual reference) is illustrated in each frame to facilitate discerning the changes in the field of view across the illustrated frames.

In each iteration I, II, III, etc., of the training routine, the model 300 makes a prediction 303 of the location of the tracking point 301 in the unmarked input frame 302, such as in any of a variety of known manners. As may be appreciated, the prediction 303 in the first iteration I is typically inaccurate, and, even more typically, is far off from the tracking point 301. However, with each subsequent iteration II, III, etc., of the training routine, the prediction 303 should increase in accuracy (e.g., as a result of updates in the parameters of the model 300 made at the end of a previous iteration I, II, III, etc., such as to be described).

The prediction 303 is compared with the ground truth 304 (the true location of the tracking point, as indicated by the medical professional’s annotations in the training dataset) to calculate the difference between the prediction 303 and the ground truth 304, and thereby to quantify the error in the prediction 303. In some aspects, the ground truth 304 is a frame, in the training routine’s dataset of ground truths, corresponding to the frame of the input frame 302 and the prediction 303, but annotated (e.g., with a medical professional’s annotations serving as ground truth data, such as described above) to indicate the actual or correct or true location of the tracking point 301 in the input frame 302 (and prediction 303 frame and ground truth 304 frame).

The ground truth 304 may serve as a benchmark or reference against which the performance of the model 300 and the accuracy of the prediction 303 are evaluated. In particular, the loss function 305 of the training routine calculates the error 306 in the prediction 303, and the error 306 is then used by the optimization algorithm of the model 300 to update the parameters (e.g., weights and/or biases) of the model 300. In some aspects, the error 306 is output in the form of the difference in the predicted coordinates of the array of pixels of the tracking point 301 and the actual coordinates of the array of pixels of the ground truth 304, each pixel having its own value (e.g., for red, green, blue, etc.).

After each iteration I, II, III, etc., of the training routine, the error 306 in the prediction 303 is calculated by the loss function 305 and applied by the algorithm to update the parameters of the model 300. As may be appreciated, any of a variety of optimization algorithms (e.g., backward propagation of errors, or backpropagation for short; gradient descent; or other optimization algorithms such as known to those of ordinary skill in the art, and/or any variations or combinations thereof, such as generally involving manipulation of, such as multiplying or adding, the pixel data) may be used to train the model 300 of an image processor 120 of the system 100 of the present disclosure to minimize the error in predicting movements of points of interest across frames of a video. In each iteration I, II, III, etc., the training routine adjusts the parameters of the model 300 to make more appropriate corrections when generating the next prediction 303. For instance, the algorithm may iteratively adjust one or more model parameters (e.g., hyperparameters such as optimizers, learning rate, batch size, etc.) to generate predictions that become increasingly closer to the ground truth with each iteration I, II, III. After multiple iterations I, II, III, etc., of a training routine, such as described above, the model 300 is modified, fine-tuned, and trained to accurately identify the location of a point, line, or region in the field of view of a medical instrument as the medical instrument is moved by a medical professional during a medical procedure. Errors decrease with each iteration I, II, III, etc., until, ultimately, the model 300 may be considered trained. The trained model has a degree of accuracy and speed necessary to successfully assist the medical procedure in performing the medical procedure.

In some aspects, if the tracking point 301 may no longer be present in the input video frame 302 during training. In such instances, the model 300 may be trained to identify the direction in which the tracking point 301 is located relative to the field of view presented in the prediction frame 303.

In some aspects, a different frame is used in each iteration I, II, III, etc., so as not to overfit the model to one training example. In some aspects, the iterations I, II, III, etc., of the training routine process sequential images (e.g., sequential frames from a batch of frames of a training video) so that the model 300 does not overfit or inappropriately generalize based on a single image or frame from the training video. Additionally or alternatively, various other parameters (e.g., hyperparameters, such as video regions or bounding boxes specified from frame to frame, number of pixels tracked, the size of the anatomical region being tracked, etc.) may be tuned or adjusted to optimize training of the model.

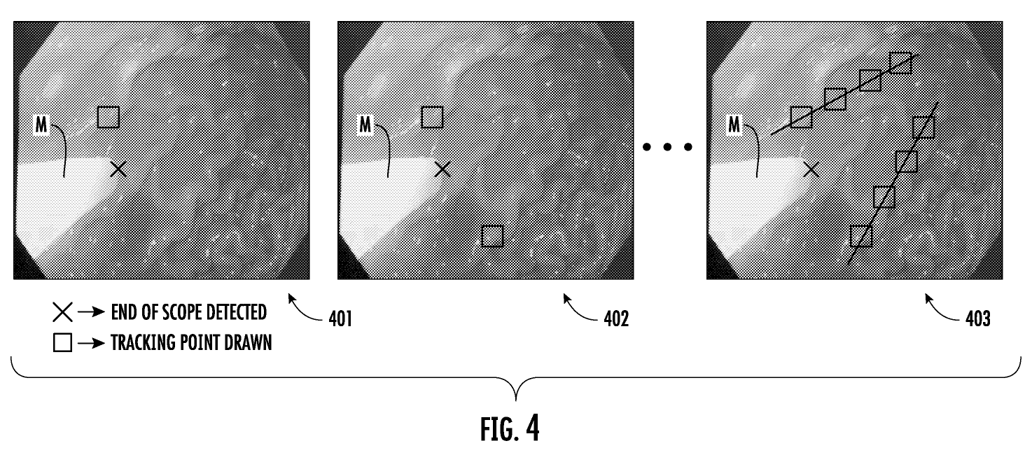

Once the tracking program or model of an image processor of a system formed in accordance with various principles of the present disclosure has been sufficiently trained, the system is able to make accurate predictions across different scenes and movements of the anatomy, and to effectively assist the medical professional in performing a medical procedure in which the medical professional has little to no direct visibility of the treatment site, and must rely on images such as digital or video images. The final system, as presented for use by a medical professional to perform a procedure, has a processor, such as an image processor, and optionally further processors and hardware operably associated with the image processor, configured with algorithms and code instructions stored on a memory or other computer readable medium and executable by one or more processors such as hardware incorporated into the system, such as to generate a graphic user interface (GUI) usable by the medical professional in performing a medical procedure using the system. A system of the present disclosure may be used in a variety of manners to virtually track points or regions along a treatment site during performance of a medical procedure, and optionally to assist in performing the procedure by predicting appropriate guidelines for achieving the desired end result of the procedure, such as schematically illustrated in FIGS. 4-8.

In accordance with the method schematically illustrated in FIG. 4, an automated system such as described herein may be used to assist with tracking points, lines, regions, etc., along the treatment site, which may otherwise be difficult to track during the procedure because of any of the above-described complexities. Instead of physically marking the anatomical structure (e.g., tissue) at the treatment site, an automated system formed in accordance with various principles of the present disclosure may allow the medical professional to digitally mark points, lines, regions, etc., along the treatment site, such as to indicate guidelines for suturing needed to perform a procedure such as an ESG procedure. For instance, the medical professional may be presented with a video frame at the beginning of the procedure. In an ESG procedure, once the stomach has been insufflated and the medical scope positioned at or near the treatment site, the medical professional may be provided with a video frame 401, such as illustrated in FIG. 4, representing the treatment site, and may make an initial digital marking (illustrated by a box). The medical professional may digitally / virtually mark one or more points, lines, regions, etc., in any of a variety of manners, including, without limitation, tracing a tip (indicated by the X in FIG. 4) of a virtual marking device M (e.g., the medical scope, or an instrument passed through the working channel of the medical scope, such as a tissue grasper, as described above with reference to FIG. 2) along the treatment site to mark the video frame 401, as indicated by the box in FIG. 4. Additionally or alternatively, the medical professional may make markings via a user interface (e.g., a button) on the medical scope to mark points digitally; and/or by inputting the points, lines, regions, etc., via a user interface (e.g., keyboard); and/or by drawing the points, lines, regions, etc., manually on a touch screen. The medical professional may, in the next frame 402, make a further marking (illustrated by another box), spaced from the box of the initial video frame 401 and generally aligned with the X indicating the tip of the medical device used to make the marking. Additional markings may be made virtually to mark points, lines, or regions to be tracked by the system of the present disclosure. The virtual markings made by the medical professional may be displayed in any of a variety of manners (optionally selectable by the medical professional from a menu of options), the box illustrated in frames 401, 402, and 403 of FIG. 4 being only one example. The final demarcations made by the medical professional are illustrated schematically in frame 403 as a line (e.g., a line of points demarcated as boxes in frames 401, 402, and generally indicated as a line in frame 403, or a continuous line made by the medical professional). The point tracking programs or models of the image processor can then be leveraged to digitally track the points, lines, regions, etc., as the landscape of the treatment site is altered by the procedure, and to display (e.g., on the visual display of the system) the points, lines, regions, etc., as they move during the procedure. In some aspects, the points, lines, regions, etc., are displayed with reference to the medical scope or other instrument being used, such as illustrated in frames 402 and 403. The end of the virtual marking device M is illustrated as an X, but other symbols, etc., may be used and may optionally be selectable by the medical professional from a menu of options. The tracked points, lines, regions, etc., may be depicted by a bounding box, pixel segmentation, pixel masking, or any other indicia useful for the medical professional (and optionally selectable from a menu). Such virtual marking may be in lieu of physical marking of the treatment site, such as previously required, and generally requires fewer medical instruments, is less invasive, and is less time-consuming. The virtual representations of the markings made by the medical professional are accurately followed by the automated system of the present disclosure, and are presented virtually on the display unit of the system.

Additionally or alternatively, the medical professional may use an automated system formed in accordance with various principles of the present disclosure to track suture points at which the medical professional has determined sutures should be placed during the procedure to achieve the desired results. In such method, the schematic illustration of FIG. 4 would indicate the points at which the medical professional intends to suture the tissue in the video frame. The initial video frame 401 may present to the medical professional a map of the initial features of the treatment site. As the medical professional sutures the tissue at the treatment site, the landscape of the treatment site is altered, with the tracking program or model maintaining each of the virtually displayed intended suture points at the correct location along the treatment site at which the medical professional initially placed the suture point. The intended suture points are illustrated in FIG. 4 as indicated by the box in frame 402, and the suture lines (which may indicate discrete points) in frame 403. However, it may be appreciated that other representations may be used, such as a symbol, shape, character, number, etc., selectable by the medical professional from a menu. In some aspects, the next point to be sutured may be the only point displayed in a frame, or may be displayed in a manner (e.g., shape, color, number, etc.) different from the other displayed points. The automated system of the present disclosure thus enables better precision for the medical professional in suturing subsequent points.

It will be appreciated that the present disclosure may use various algorithms or programs or models, in addition to (or instead of) those described with reference to FIG. 4, to track points, lines, regions, etc. Typically, the algorithm / program / model is a one-shot point, line, or region tracking algorithm / program / model in which one video frame with a point, line, or region marked on the frame is provided. This video frame is provided to the algorithm / program / model, which subsequently tracks the point, line, or region in subsequent video frames. The algorithm / program / model must be capable of handling the various challenges presented by the procedure, such the challenges described above. Various suitable algorithms which may be used include, without limitation, optical flow trackers (e.g., Lucas-Kanade, Farneback Optical Flow); Correlation Filter Trackers (e.g., Kernelized Correlation Filters (KCF), Discriminative Scale Space Trackers (DSST), etc.), such as for optimal real-time performance; Siamese Network Trackers (e.g., Siamese Fully Convolutional (SiamFC), Siamese Region Proposal Network trackers (SiamRPN), etc., such as for optimal accuracy, but which may require more computational requirements); Particle Filter Trackers (e.g., Conditional Density Propagation Trackers (CONDENSATION)); or other suitable trackers, including combinations thereof.

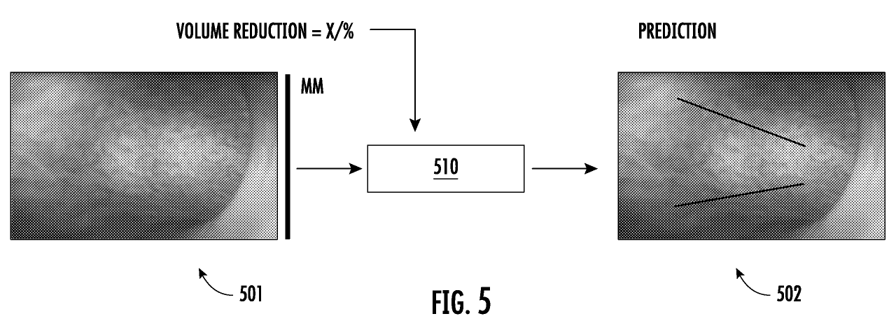

An automated system formed in accordance with various principles of the present disclosure may optionally be used to provide automated predictions and/or suggestions, such as an alternative to input by the medical professional. An example of an embodiment of a method of guideline suggestion / prediction by the automated system of the present disclosure is illustrated schematically in FIG. 5. The automated system is presented with the landscape of the treatment site in the initial video frame 501 (e.g., an image of the insufflated stomach). The medical professional then inputs (e.g., via a user interface 140 such as described above with reference to FIG. 1) the desired outcome. For instance, the medical professional may input a desired percent decrease of / reduction in the stomach volume. The processor 510 (e.g., image processor) of the automated system includes programs (e.g., neural networks) which can be used to predict suture lines and/or the points along which the medical professional must suture the stomach to achieve the desired volume reduction, such as illustrated in frame 502. Suture points displayed in accordance with this example of a method may be displayed in any of the above-described ways with reference to the methods schematically illustrated by FIG. 4, reference being made thereto for brevity and without intent to limit. The displayed guidelines (suture points, lines, etc.) are automatically altered to conform to the continually changing contours of the patient’s anatomy. Thus, the continually changing contours of the treatment site, such as the stomach, no longer present challenges to the medical professional as the medical professional may follow the guidelines generated and displayed by an automated system formed in accordance with various principles of the present disclosure.

Various programs / models may be used for tracking as described with reference to the method schematically depicted by FIG. 5. For instance, a Neural Network, a Convolutional Neural Network, and/or a Deep Neural Network may be used for predicting guidelines given an image of the treatment site (e.g., an image of an insufflated stomach) and the intended result (e.g., percent internal volume reduction of the stomach). The program / model may initially be trained on a collection of datasets, such as, without limitation, images of a treatment site (e.g., an insufflated stomach), suggested and/or annotated guidelines on an image, and a final result of following the guidelines (e.g., an image of how much the stomach volume decreased post-procedure). The ground truth dataset may be created by a medical professional’s validation of volume reduction resulting from suturing along a given set of lines in an initial video frame of the training video. The training input may include images (e.g., with the guidelines removed), and the desired outcome (e.g., value of percent volume reduction), and the output would be the predicted guidelines compared to the ground truth image with the guidelines annotated by a medical professional.

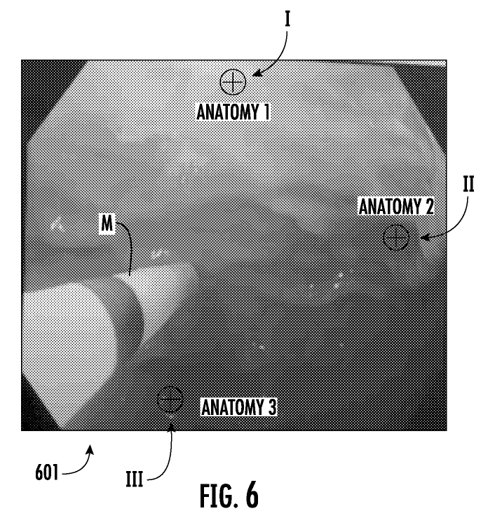

Yet another method of using an automated system such as formed in accordance with various principles of the present disclosure includes anatomical mapping of and/or creation of a compass for a treatment site. In the example of an embodiment schematically illustrated in FIG. 6, the automated system may be used to generate what may be referenced as an anatomical compass. Such anatomical compass may be used to assist a medical professional in navigating with a patient’s anatomy, particularly as the anatomy is changed by the procedure the medical professional is performing. The automated system undergoes anatomical mapping training to learn to maintain relative positions of tracked points even when some of those points move off-screen (out of the range of the visual display). A video frame displaying the treatment site may be presented to the medical professional to mark anatomical landmarks (e.g., at or near the treatment site), directions, etc., such as points I¸ II, III schematically illustrated in FIG. 6. For instance, in an ESG procedure, once the stomach has been insufflated and the medical scope positioned at or near the treatment site, the medical professional may be provided with a video frame 601 representing the insufflated stomach. The medical professional may indicate anatomical landmarks (e.g., the pylorus, fundus, cardia, lesser curvature, greater curvature, etc., of the stomach), and/or anatomical directions (e.g., proximal, distal, lateral directions), or other points, lines, regions, etc., of interest, such as which the medical professional may want to reference during the procedure (but which may change position, orientation, configuration, etc., as the procedure progresses). The points, lines, regions, etc., may be marked and tracked in a manner such as in the methods described with reference to FIG. 4, reference being made thereto for brevity and without intent to limit. In some aspects, the medical professional marks the points, lines, regions, etc., such as with a virtual marking device M, prior to commencing the medical procedure. In some aspects, the medical professional may mark the points, lines, regions, etc., while performing the medical procedure (e.g., to track a point of interest noted during the procedure). The image processor of the automated system of the present disclosure then stores such points, lines, regions, etc., and analyzes associations between such points, lines, regions, etc., to track these points, lines, regions, etc., and to be able to maintain a virtual display of such points, lines, regions, etc., as the procedure progresses. Because a given point or location along the anatomy moves (laterally, rotationally, etc.) as the anatomy is reconfigured, remodeled, etc., such anatomical mapping assists in orienting the medical professional to allow the medical professional to know the direction in which he/she is to move, and/or the location to which he/she is to move a medical instrument and/or to continue performing the procedure. Such mapping thereby enables the medical professional to achieve greater precision in performing the procedure, such as greater precision in determining subsequent suture points. In some aspects, the suture points may be digitally tracked, with the automated mapping orienting the medical professional with respect to the patient’s anatomy in general, and with respect to the treatment site in particular.

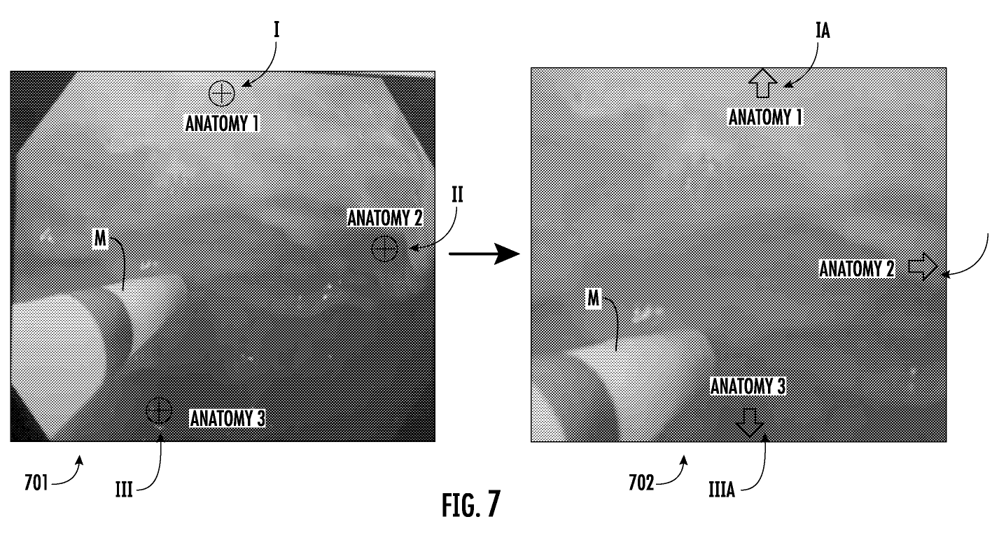

In addition to or instead of tracking and displaying anatomical landmarks, directions, etc., as described above with reference to FIG. 6, an automated system formed in accordance with various principles of the present disclosure may be used to generate a directional indicator for the medical professional to use in performing a procedure within a patient’s body. For instance, as illustrated schematically in FIG. 7, a video frame 701 (e.g., an image of an insufflated stomach) may be presented to the medical professional to mark anatomical landmarks, directions, etc., such as points I¸ II, III, such as in any of the manners described herein. Additionally or alternatively, the medical professional may mark landmarks, directions, etc., while performing the medical procedure (e.g., to track a point of interest noted during the procedure). A virtual marking device M may be used to mark the landmarks, directions, etc., such as described above with reference to FIG. 4 and FIG. 6. Because anatomical structures such as the stomach present a continuous surface to the imaging device, the image processor can extrapolate and predict / indicate where a point along the anatomical structure is located even if the point is not within the video frame. The image processor may be used to generate directional arrows IA¸ IIA, IIIA, such as illustrated in video frame 702, pointing to a point, line, or region, etc., of interest which may have moved off-screen (out of the range of the visual display) and/or indicating the direction in which the no-longer-visible point is located. Thus, based on the surface being visualized, and the points which have been tracked (such as in any of the above-described manners, reference being made thereto for the sake of brevity and without intent to limit), once a point, line, or region, etc., of interest travels off-screen, the medical professional is provided with an arrow or indicator, such as along the border of the video frame, to be guided to such point, line, or region, etc., of interest.

The program / model training which may be used to perform a mapping method as described with reference to FIG. 6 and/or FIG. 7 incorporates the creation of ”maps” tracking multiple points on deformable surfaces to predict off-screen point locations based on visible reference points. For instance, associations between points of interest on-screen may be stored relative to each other. As some points move off-screen, points which remain on-screen can be used to predict the direction in which the medical professional must move to get back to points currently off-screen. Essentially, a “map” of points on a deformable surface is created and tracked frame-to-frame.

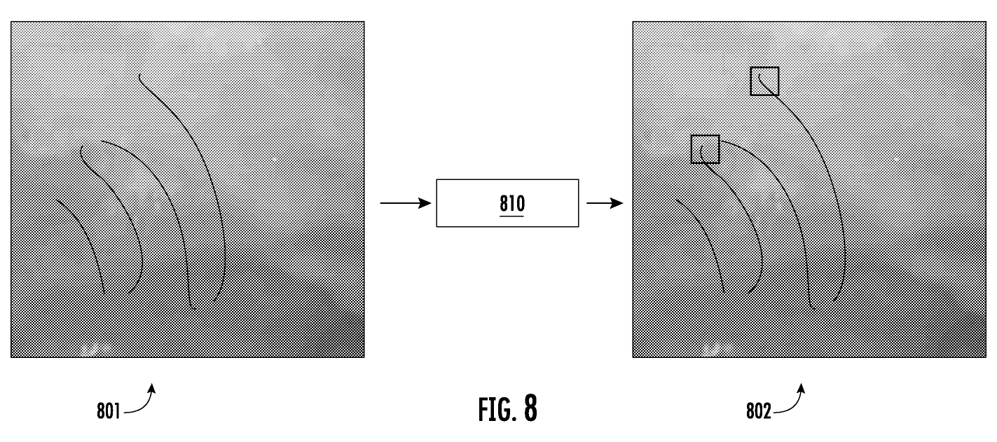

Yet another method of using an automated system such as formed in accordance with various principles of the present disclosure is illustrated in FIG. 8, schematically indicating detecting undesirable and/or unintended structural configurations (e.g., of the anatomy and/or a structure with respect to the anatomy) which may occur during the procedure, such as suture kinks. For instance, the program / model may be trained to recognize an appropriate configuration of a suture (without any kinks) as it is positioned and cinched within a body, in contrast with a suture with a kink. Additionally or alternatively, the program / model may be trained to predict the outcome of a given procedure, such as the outcome of a suturing pattern recommended by the program / model. Configurations of sutures which do not meet the predicted configuration, such as a suture with a kink, such as illustrated in frame 801, and/or a suture pattern not having a predicted configuration may be detected and a notification generated to alert the medical professional, such as to take appropriate action. In some aspects, the program / model may detect potential kinks in a suture before, during, and/or after cinching of the suture, such as based on the movement and/or bending pattern of the suture. Notifications or alerts by the automated system, such as a visual image (e.g., a box around the area of the alert) such as illustrated in frame 802, or an audio alert (e.g., word, sound, etc.), or otherwise, may assist the medical professional in taking the appropriate action for an event which may have been difficult to visualize otherwise.

Various programs or models may be used to detect undesirable and/or unintended configurations. For instance, a Neural Network, a Convolutional Neural Network, and/or a Deep Neural Network may be used for detecting and/or localizing an undesirable and/or unintended configuration, such as a kink in a suture or a suture crossing which could lead to a kink. To train program / model to detect undesirable and/or unintended structural configurations of anatomy or structures implanted with respect to the anatomy, the program / model may be presented with numerous examples of acceptable configurations and undesirable configurations. For instance, the program / model may be presented with numerous images of sutures with and without kinks. The above-described frame 801 displaying sutures with kinks may be presented to the program / model for training purposes. The image details are input into the program / model 810, with ground truth annotations such as frame labels, bounding boxes, pixel segmentation, etc., marking kink locations, such as illustrated schematically in FIG. 8. Typically, a medical professional provides annotations, such as by annotating video frames, for initial input as well as during program / model development and training. Frame 802 in FIG. 8 may represent indications of predictions (e.g., the illustrated boxes) output by the program / model 810, such as based on training using annotations performed by qualified medical professionals. Multiple iterations of training may be performed to enhance accuracy, such as described above with reference to various training of programs / models described herein, or as known to those of ordinary skill in the art.

As may be appreciated in view of the above, an automated system formed in accordance with various principles of the present disclosure leverages various machine learning techniques to track points or patterns, and/or to make predictions and/or to provide guidelines. Various pre-existing programs or models are trained in accordance with various principles of the present disclosure, and fine-tuned with proprietary medical imaging datasets to achieve the required accuracy for clinical use.

It will be appreciated that the programs or models, training of programs or models, and/or ground truth dataset used in training and developing programs or models in accordance with various principles of the present disclosure may use a computer-readable storage medium in the form of any non-transitory computer-readable storage medium or machine-readable storage medium, such as an optical, magnetic or semiconductor storage medium. The terms “machine-storage medium,” “device-storage medium,” “computer-storage medium” may be usable interchangeably herein, such as to refer to a single or multiple storage devices and/or media (e.g., a centralized or distributed database, and/or associated caches and servers) that store executable instructions and/or data. The terms shall accordingly be taken to include, but are not be limited to, solid-state memories, and optical and magnetic media, including memory internal or external to processors. Specific examples of machine-storage media, computer-storage media and/or device-storage media include, without limitation, non-volatile memory, including, by way of non-limiting examples, semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks.

The computer-readable storage medium may store computer executable instructions with which circuitry (e.g., processor, or the like, such as known in the art) can execute. For example, computer executable instructions can include instructions to implement operations described with respect to logic flow, ground truth data, instructions, guidance, or otherwise. Examples of computer-readable storage medium or machine-readable storage medium may include any tangible media capable of storing electronic data, including, without limitation, volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like.

It will be that the image processor described herein may be in the form, or a component of, a computer system within which a set of instructions may be executed for causing the image processor to perform any one or more of the methodologies discussed herein. For instance, instructions such as software, a program, an application, an applet, an app, or other executable code, may be executed to cause the processor to perform or execute any one or more of the methodologies discussed herein. For example, the instructions may cause the image processor to execute predictions; track point, lines, or regions; generate recommendations, etc.; and/or to perform other operations such as discussed herein. The image processor may include, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), other smart devices, a web appliance, a network router, a network switch, a network bridge, or any machine capable of executing the instructions, sequentially or otherwise, that specify actions to be taken by the machine. The image processor may operate as a standalone device or may be coupled (e.g., networked) to other processors / machines. Further, while reference is made to an “image processor”, such reference shall also be taken to include a collection of processors that individually or jointly execute the instructions to perform any one or more of the methodologies discussed herein. The image processor, or associated processors, may include (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof). The term “processor” is intended to include a single processor with a single core, a single processor with multiple cores (e.g., a multi-core processor), multiple processors with a single core, multiple processors with multiples cores, multi-core processors that may comprise two or more independent processors (sometimes referred to as “cores”) that may execute instructions contemporaneously, and/or any combination thereof.

In addition, an image processor used herein may include memory and I/O components which may be configured to communicate with each other such as via a bus. The memory may include a main memory, a static memory, a processor memory, and/or a storage unit, which may store instructions embodying any one or more of the methodologies or functions described herein, and may be accessible to the image processor such as via a bus. The various memories may store one or more sets of instructions and data structures (e.g., software) embodying or utilized by any one or more of the methodologies or functions described herein. These instructions, when executed, cause various operations to implement the disclosed operations of a visualization system as described herein. Instructions utilized by the image processor may reside, completely or partially, within the main memory, the static memory, machine-readable medium within the storage unit, at least one of the associated processors (e.g., within the image processor’s cache memory), and/or any suitable combination thereof, during execution thereof by the image processor.

The I/O components may include a wide variety of components to receive input, provide output, produce output, transmit information, exchange information, capture measurements, and so on. The specific I/O components that are included in a particular image processor will depend on the type of image processor. In some aspects, the I/O components may include output components and input components. The output components may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like. In some aspects, the I/O components may include biometric components, motion components, environmental components, and/or position components, among a wide array of other components. For example, biometric components may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. Motion components may include acceleration sensor components (e.g., accelerometer), gravitation sensor components, rotation sensor components (e.g., gyroscope), and so forth.

It will be appreciated that although the present disclosure is described with specific reference to ESG procedures, it should be appreciated that the medical systems and methods described herein may be used for other procedures as well. Moreover, although the present disclosure is described with reference to treating the gastrointestinal system, it should be appreciated that such medical systems and methods may be used to treat tissues of the abdominal cavity, digestive system, urinary tract, reproductive tract, respiratory system, cardiovascular system, circulatory system, and the like.

It is to be understood by one of ordinary skill in the art that the present discussion is a description of illustrative examples of embodiments only, and is not intended as limiting the broader aspects of the present disclosure. Accordingly, although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. Various further benefits of the various aspects, features, components, and structures of systems and methods such as described above, in addition to those discussed above, may be appreciated by those of ordinary skill in the art.

It will be appreciated that all systems and methods discussed herein are examples of systems and/or methods implemented in accordance with one or more principles of this disclosure. These examples are not the only way to implement these principles but are merely examples, not intended as limiting the broader aspects of the present disclosure. Other examples of manners of implementing the disclosed principles will occur to a person of ordinary skill in the art upon reading this disclosure. It should be apparent to those of ordinary skill in the art that variations can be applied to the disclosed systems and/or methods, and/or to the sequence of steps of the methods described herein without departing from the concept, spirit, and scope of the disclosure. It will be appreciated that various features described with respect to one embodiment may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. While operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An automated system for assisting with performance of a medical procedure with respect to a treatment site along the anatomy of a patient, the system comprising:
an image processor operably associated with a medical instrument having an imaging device, the image processor configured to receive and process signals from the imaging device to identify a point, line, region, or object along a patient’s anatomy, and to predict movement of the point, line, region, or object along the patient’s anatomy as the patient’s anatomy is modified during the course of a medical procedure; and
a video display unit operably associated with the image processor to display the point, line, region, or object to the medical professional in real-time while the medical professional performs the medical procedure.

2. The system of claim 1, wherein the image processor is configured to output a representation of markings input by the medical professional.

3. The system of claim 1, wherein the image processor is configured to output a representation of markings of one or more lines of sutures the medical professional intends to make along the anatomy of the patient, and to track the representation of the markings as the medical professional performs the medical procedure.

4. The system of claim 1, wherein the image processor is configured to output a representation of points at which the medical professional intends to suture along the treatment site.

5. The system of claim 1, wherein the image processor is configured to output a representation of directions and/or landmarks to orient the medical professional as the patient’s anatomy is modified during the course of the medical procedure.

6. The system of claim 5, wherein the output of the image processor is based on markings input by the medical professional indicating anatomical directions at the treatment site and/or anatomical landmarks at or near the treatment site.

7. The system of claim 5, wherein the output of the image processor is an indication of a direction in which a point, line, or region of the anatomy is located, the point, line, or region not being visually displayed by the video display unit.

8. The system of claim 7, wherein the point, line, or region is input by the medical professional prior to commencing the procedure and/or during the medical procedure.

9. The system of claim 1, wherein the image processor is configured to output an indication of an undesirable and/or unintended configuration occurring during the medical procedure.

10. The system of claim 9, wherein the output of the image processor is an indication of a kink in a suture used during the medical procedure.

11. An automated system for assisting with surgical procedures within a patient’s stomach, the system comprising:
an image processor operably associable with an imaging device of an endoscope and configured to receive and process signals from the imaging device to identify a point, line, region of the patient’s stomach and to predict movement of the point, line, or region, or an object along the patient’s stomach as the configuration of the patient’s stomach is modified during the course of a medical procedure; and
a video display unit operably associated with the image processor to display the point, line, region, or object to the medical professional in real-time while the medical professional performs the medical procedure.

12. The system of claim 11, wherein the image processor is configured to output a representation of markings of one or more lines of sutures the medical professional intends to make along the patient’s stomach, and to track the representation of the markings as the medical professional performs the medical procedure and the contours of the stomach are modified by the medical procedure.

13. The system of claim 11, wherein the image processor is configured to output a representation of points at which the medical professional intends to suture along the stomach to modify the shape or volume of the stomach, and to track the representation of the points as the medical professional performs the medical procedure and the contours of the stomach are modified by the medical procedure.

14. The system of claim 11, wherein the image processor is configured to output a representation of directions and/or landmarks within the patient’s stomach to orient the medical professional as the patient’s stomach is modified during the course of the medical procedure, and/or to direct the medical professional in the direction and/or landmark if not represented on the video display unit.

15. The system of claim 11, wherein the image processor is configured to output an indication of a kink in a suture and/or an undesirable and/or unintended configuration of the stomach occurring during the medical procedure.

16. A method of providing automated assistance during performance of an endoscopic sleeve gastroplasty procedure, the method comprising:
providing an image processor trained to track points, lines, regions, or objects, along a stomach lining of a patient and operably associable with an imaging device of an endoscope; and
providing a video display unit for displaying images from the imaging device of the endoscope processed by the image processor, including virtual representations of points, lines, regions, or objects of interest to the medical professional for performing the endoscopic sleeve gastroplasty procedure.

17. The method of claim 16, further comprising providing a user interface for the medical professional to input points, lines, or regions of interest to track and to display on the video display unit during the procedure.

18. The method of claim 16, wherein providing an image processor further comprises providing an image processor trained to predict and recommend guidelines for suturing the patient’s stomach to reduce the volume of the patient’s stomach.

19. The method of claim 16, wherein providing an image processor further comprises providing an image processor trained to notify the medical professional of a kink in a suture and/or an undesirable and/or unintended configuration of the stomach occurring during the endoscopic sleeve gastroplasty procedure.

20. The method of claim 16, wherein providing an image processor further comprises providing an image processor trained to indicate anatomical directions and/or landmarks within the stomach as the configuration of the stomach changes during the endoscopic sleeve gastroplasty procedure.
